(19) United States
(12) Patent Application Publication
(10) Pub. No.: US 2005/0187423 A1
(43) Pub. Date: Aug. 25, 2005

(54) Magnetic therapeutic device

(76) Inventors: Vincent Ardizzone, Port Jefferson, NY (US); Thomas Bove, Spokane, WA (US)

Correspondence Address: CISLO & THOMAS, LLP, 233 WILSHIRE BLVD, SUITE 900, SANTA MONICA, CA, 90401-1211, US

(21) Appl. No.: 11/040,517

(22) Filed: Jan. 21, 2005

Related U.S. Application Data
(63) Continuation-in-part of application No. 10/318,552, filed on Dec. 13, 2002, now abandoned.

(51) Int. Cl. A61B 17/52; A61N 2/00

(52) U.S. Cl. 600/9

(57) ABSTRACT

A magnetic therapeutic device comprises a ring-shaped housing adapted to revolve about a first axis of rotation. The therapeutic device also comprises a spherical magnetic member adapted to revolve about a second axis of rotation within the ring-shaped housing while the housing revolves about the first axis of rotation. The spherical magnetic member simultaneously revolves about the first and second axes to provide therapeutic massage-like effects to a treatment area.

### CROSS-REFERENCE TO RELATED APPLICATIONS

This application is a continuation-in-part of U.S. patent application Ser. No. 10/318,552, filed Dec. 13, 2002, which claims the benefit of U.S. Pat. No. 6,648,812, issued Nov. 18, 2003, which claims the benefit of U.S. Provisional Patent Application Ser. No. 60/272,384, filed Feb. 28, 2001. These applications and issued patent are incorporated by reference as if fully stated herein.

### BACKGROUND

Various devices have been made to create time-varying magnetic fields for use on the human body. Generally, two types of time-varying magnetic fields have been used. The first type used an alternating current (“AC”) field that is produced when electric current is caused to alternate at any given frequency. In accordance with Maxwell's equations, a magnetic field is concurrently produced at the same frequency as the electric field. Included in this first type of time-varying magnetic field device are pulsed electromagnetic fields (PEMF) which are generated when a current is caused to move through a conductor in discrete impulses of electric charge moving in the same direction.

A second general type of device for creating time-varying magnetic fields involves physically moving a static magnetic field through space. While linear displacement is one way to accomplish this, another common method involves rotating the static magnetic field. The source of the static magnetic field is generally a permanent magnet, since an electromagnet requires considerable expenditure of energy in the form of current generation and the subsequent dissipation of unwanted heat energy.

The therapeutic uses of time-varying magnetic fields have been described and clinically evaluated in the literature. The more popular publications written for the general public include “Magnetic Therapy” by Dr. Ronald Lawrence and Dr. Paul Rosch, “The Pain Relief Breakthrough” by Dr. Julian Whitaker and Brenda Adderly, and “Magnetic Therapy in Eastern Europe” by Dr. Jiri Jerabek and Dr. William Pawluk. These books offer numerous references to clinical studies which purport to show the effectiveness of time-varying magnetic fields for the treatment of a multitude of chronic and acute conditions including atherosclerosis, carpal tunnel syndrome, chronic bronchitis, post-ischemic injury, edema, fractures, infected wounds, limb grafts, burns, scars, macular degeneration, etc. The lack of any substantial negative side effects is also purported for most treatments. In recent years, the general public and even the medical community have increasingly accepted magnetic therapy as an alternative treatment worthy of consideration for such conditions.

Patented devices, which utilize permanent magnets to produce a time-varying magnetic field for therapeutic purposes, include Horl U.S. Pat. No. 4,727,857; Kleitz U.S. Pat. No. 5,632,720; and Souder U.S. Pat. No. 6,001,055. All of these devices function by causing permanent magnets to rotate around a fixed axis. The magnetic field generated by each of these devices sweeps out into space in a single direction. Changing the angle of the rotation requires manual manipulation of the entire device since the axis upon which the magnets rotate is stationary. It has been observed that the angle at which magnetic flux lines cut through tissue can influence the degree of beneficial effects. What is needed therefore is a handheld device or a device capable of being attached to a part of the body or clothing, or the like, that will create a sweeping magnetic field in a multitude of directions, thus providing more complete angular coverage to the part of the body being treated with the moving magnetic field.

### SUMMARY

Exemplary embodiments disclosed herein are generally directed to a magnetic therapeutic device.

In accordance with one aspect of the invention, the magnetic therapeutic device comprises a housing adapted to revolve about a first axis of rotation, and at least one magnetic member adapted to revolve about a second axis of rotation within the housing while the housing revolves about the first axis of rotation. The magnetic member simultaneously revolves about the first and second axes to provide therapeutic massage-like effects to a treatment area.

This and other aspects of the invention will become apparent from a review of the accompanying drawings and the following detailed description of the invention.

### BRIEF DESCRIPTION OF THE DRAWINGS

The invention is generally shown by way of reference to the accompanying drawings in which:

FIG. 1 is a top view of one embodiment of the present invention;

FIG. 2 is a front view of one embodiment of the present invention;

FIG. 3 is a perspective view of another embodiment of the present invention;

FIG. 4 is a perspective view of another embodiment of the present invention;

FIG. 5 is a perspective view of a cover of one embodiment of the present invention;

FIG. 6 is a perspective view of another embodiment of the present invention;

FIG. 7 is a perspective view of another embodiment of the present invention;

FIG. 8 is a perspective view of another embodiment of the present invention showing a magnetic unit and a free moving member;

FIG. 9 is a perspective view of the embodiment shown in FIG. 8 further showing an enclosure assembly and a retainer member;

FIG. 10 a perspective view of the embodiment shown in FIG. 9 from a different angle showing an annular rolling surface and a pivot member;

FIG. 11 is a perspective view of the embodiment FIG. 8 further showing a DC motor and right-angle gear box attached to the magnetic unit and a partially disassembled cover for enclosing the invention;

FIG. 12 is a perspective view of the embodiment FIG. 8 further showing how the component parts fit in a cover;

FIG. 13 is a perspective view of the embodiment shown in FIG. 12 from a different angle;

FIG. 14 is a perspective view of a cover for the embodiment shown in FIG. 8;

FIG. 15 is a perspective view of a cover for the embodiment shown in FIG. 14 from a different angle;

FIG. 16 is an exploded view of another embodiment of the present invention;

FIG. 17 is a partially exploded perspective view of an embodiment of the present invention showing a stationary magnet on a stationary track;

FIG. 18a is a top plan view of an embodiment of the present invention showing stationary magnets on a stationary track;

FIG. 18b is another top plan view of an embodiment of the present invention showing stationary magnets on a stationary track;

FIG. 19 is an exploded perspective view of a magnetic therapeutic device in accordance with an alternative embodiment of the present invention;

FIG. 20 is a perspective view of the magnetic therapeutic device of FIG. 19 in partially assembled state; and

FIG. 21 is a bottom perspective view of one component of the magnetic therapeutic device of FIG. 19.

### DETAILED DESCRIPTION

The detailed description set forth below in connection with the appended drawings is intended as a description of exemplary embodiments and is not intended to represent the only forms in which the exemplary embodiments may be constructed and/or utilized. The description sets forth the functions and the sequence of steps for constructing and operating the exemplary embodiments in connection with the illustrated embodiments. However, it is to be understood that the same or equivalent functions and sequences may be accomplished by different embodiments that are also intended to be encompassed within the spirit and scope of the invention.

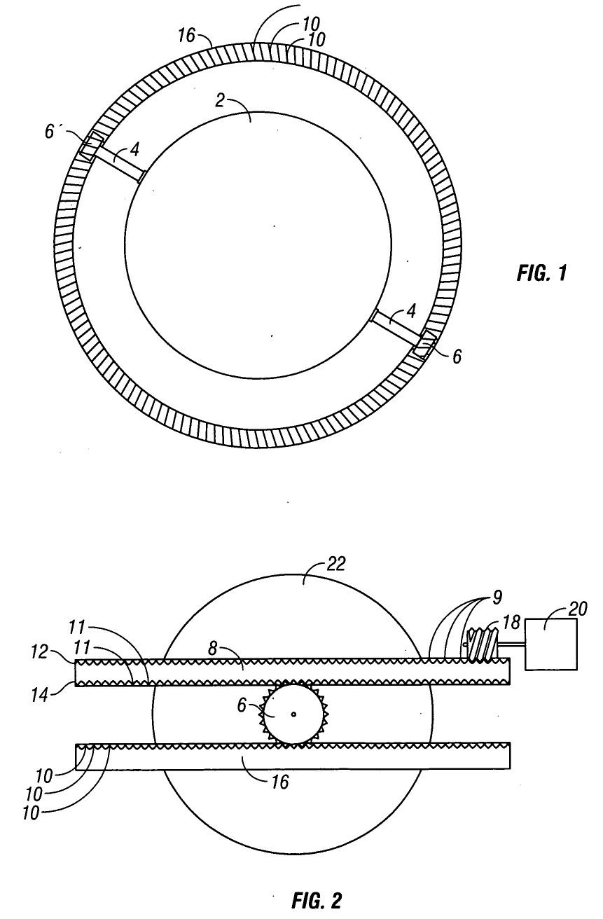

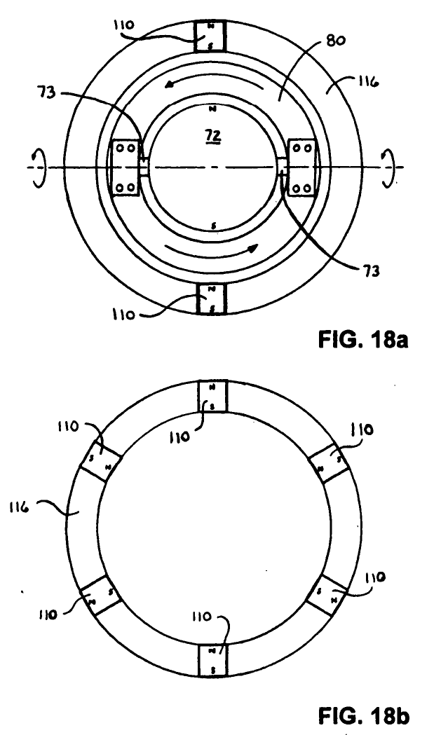

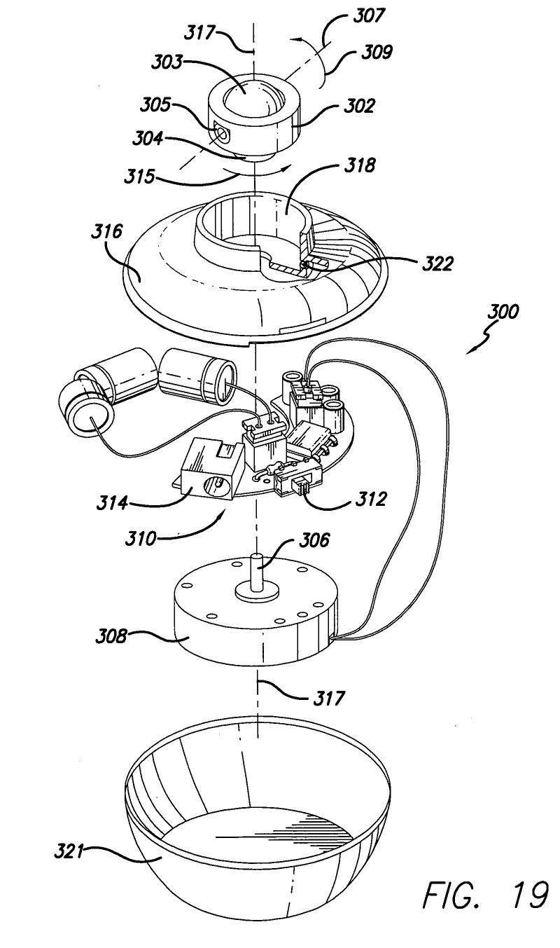

Some embodiments of the invention will be described in detail with reference to the related drawings of FIGS. 1-21. Additional embodiments, features and/or advantages of the invention will become apparent from the ensuing description or may be learned by practicing the invention. In the figures, the drawings are not to scale with like numerals referring to like features throughout both the drawings and the description.

The present invention relates generally to a magnetic field generating apparatus and more specifically to a magnetic field generating apparatus that produces a time-varying angular displacement of magnetic flux density for use in therapeutic applications on humans or animals.

In accordance with an exemplary embodiment of the present invention, as generally illustrated in FIG. 1, bi-axial rotation is accomplished by mounting a permanent magnet 2 on a rod 4 that has a drive gear 6, or other meshing or traction surface arrangement, attached at one end. Although a gear system is used in the preferred embodiment, other embodiments without the use of gears can be used, such as an o-ring, sprocket, or rubberized surface capable of imposing an angular force on the rod by contact with an external force. The magnet 2, rod 4 and drive gear 6 assembly is then placed on a rotatable circular track 8, having either matching gear teeth or other surface for exerting this external force on the ends of the rod 4. The magnet 2 can be rectangular or substantially spherical in shape.

In one embodiment, rod 4 is sandwiched between the rotatable circular track 8 and a matching stationary circular track 16, which faces the rotatable circular track 8. A motor 20 is coupled to the rotatable circular track 8 and causes the track to rotate. The angular force imparted on drive gear 6 causes rod 4 to turn with track 8. Since the drive gear 6 is engaged with the surfaces of both circular tracks 8 and 16, drive gear 6 is forced to roll at the same time it moves along the circular tracks. The drive gear 6 causes rod 4 to roll like an axle in a direction perpendicular to the direction of the rotation of rod 4 around the circular tracks 8 and 16. The magnet 2 is thereby caused to move in both a primary rotational movement and a secondary rotational movement, both turning and rolling the magnet 2. As a result, this configuration creates a complex bi-axial sweeping action of the magnetic field. As shown in FIG. 1, a placeholder or floating gear 6′ is placed on the end of rod 4 opposite drive gear 6 for balance and stability purposes. Floating gear 6′ is rotatably mounted to rod 4 so that this gear 6′ does not impart a rolling force on rod 4, and only the drive gear 6 causes rod 4 to roll.

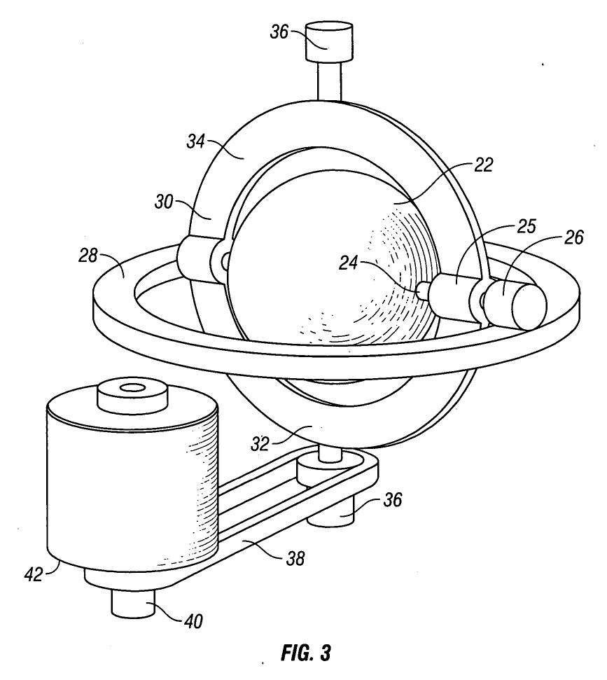

In particular, one embodiment of the present invention is shown in FIGS. 1 and 2. FIG. 1 illustrates the embodiment from a top view showing the stationary gear track 16, gear teeth 10, magnetic unit 2, the ends of rod 4, drive gear 6, and floating gear 6′. FIG. 2 shows the embodiment from a side view further showing free moving gear ring 8 and motor 20. (In FIG. 1, the free moving gear ring 8 and motor 20 have been removed for purposes of illustration of the gears 6 and 6′ and stationary gear track 16.) As shown in FIG. 1, a magnetic unit 2 is mounted to a rod 4. Although the magnetic unit 2 shown in this embodiment is spherical in shape, other shaped magnets can be used such as a bar magnet, a sheet magnet having a pre-determined magnetic pattern, or the like. Additionally, although the embodiment illustrated shows rod 4 extending through magnetic unit 2, rod 4 may alternatively attach to one side or end of magnetic unit 2.

One end of the rod 4 contains a drive gear 6 that rides between a stationary gear ring 16 and free moving gear ring 8. The other end of rod 4 contains floating gear 6′ which likewise rides between a stationary gear ring 16 and free moving gear ring 8. Preferably, the stationary gear ring 16 and the free moving gear ring 8 are made from a generally non-magnetic material so as not to interfere with the magnetic field produced by the magnetic unit 2. The free moving gear ring 8 has gear teeth 9 on a first surface 12 and gear teeth 11 on a second surface 14. Motor 20 drives gear 18, which in turn engages gear teeth 9 of the first surface 12 of ring 8, either directly or indirectly. Accordingly, as the gear 18 turns, it drives the free moving gear ring 8.

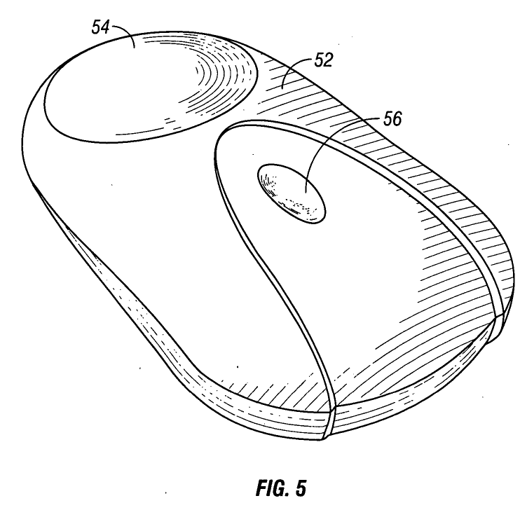

As the free moving gear ring 8 turns, the rod 4 is forced to turn, creating a primary rotational movement of the magnetic unit 2 because gears 6 and 6′ are engaged with the gear teeth 11 on the second surface 14 of the free moving gear ring 8. End gears 6 and 6′, however, also engage the teeth 10 on the surface of stationary ring 16. While floating gear 6′ is rotatably mounted to one rod 4, and thereby does not impart a rolling force on rod 4, drive gear 6 is fixed to the other rod 4. As a result, when drive gear 6 is forced to roll as it rotates along free moving gear ring 8, rod 4 is forced to likewise roll about this second axis. Hence, a bi-axial rotation of magnetic unit 2 is produced, creating a complex bi-axial sweeping action of the magnetic field. With this design, only a single magnet 2 is necessary to produce this complex time-varying magnetic field. The entire embodiment can be housed inside a plastic housing (e.g., see FIGS. 5 and 11 through 15) allowing the spherical magnetic unit 2 to rotate freely about two separate axes. The present invention can be positioned or moved by hand over a desired region of the human body, or it can be attached to a part of the user's body or clothing.

In another embodiment a magnet assembly comprises a free moving member 30 having one or more extensions surrounding a magnetic unit 22. A rod 24 is mounted to the magnetic unit 22. The two ends of the rod 24 extend beyond the free moving member 30. A rolling member 26 is fixed to one end of rod 24. (Optionally, a slipping member may be rotatably mounted to the opposite end of rod 24, it has been found that, given sufficient precision of component parts, and minimization of the tolerances involved, a second floating or slipping member is not necessary.) This embodiment includes a cover 52 that surrounds the magnet assembly and has an inner surface that defines a circumferential groove that houses the rolling member 26, and that defines one or more pivot members 79 for pivotally retaining the magnet assembly. The rod 24, the rolling member 26, the free moving member 30, and the cover 52 are all preferably made of substantially non-magnetic materials.

A motor 42 or the like is coupled to an extension 36 on the free moving member 30, either directly or indirectly using a drive belt, gear box, or the like. The turning of the motor 42 then causes the magnet assembly to rotate. As the magnet assembly rotates, the rolling member and the slipping member are forced to roll due to contact with an annular surface 28 of the circumferential groove formed in the inner surface of the cover 52. This rolling action of the rolling member 26 causes the magnetic unit 22 to roll. Thus, the magnetic unit 22 both rotates about one axis and rolls about another, producing a time-varying field of magnetic flux density and a time-varying field of angular flux displacement for use in connection with humans or animals for therapeutic purposes.

One example of this embodiment is shown in FIG. 3. As in the prior embodiment, the magnetic unit 22 is mounted to a rod 24. One end of the rod 24 has a rolling member 26, which is in contact with the annular surface 28. Surface 28 may be formed in the cover as described above and surrounds the magnetic unit 22. In FIG. 3, the rest of the cover has been removed so that only surface 28 is shown in the illustration. Surface 28 can also be a stationary ring with sufficient surface traction to exert an angular force on rolling member 26. The rolling member 26 and/or surface 28 would preferably consist of an elastomeric material or other material with sufficient gripping properties. Also, surrounding the spherical magnetic unit 22 is a free moving member 30, which is radially inside and oblique to surface 28. The rod 24 rotatably extends through openings 25 in the free moving member 30. As a result, when the rolling member 26 rotates along the surface 28, so does the free moving member 30, but the rolling action of the rolling member 26 does not cause the free moving member 30 to likewise roll.

On each of the first half 32 and the second half 34 of free moving member 30 there exists an extension 36 that is pivotally mounted to a casing or cover 52. An example of such a cover is shown in FIG. 5, and an example of a pivot member 79 for pivotally mounting the free moving member 30 within the cover 52 is shown in FIG. 10. Attached to the extension 36 on the first half 32 of the free moving ring 30 is a drive belt 38. In this embodiment, the drive belt 38 is a rubberized belt, but can be any material with similar properties, such as a toothed belt, chain, or the like. The drive belt 38 also attaches to a rotatable unit 40 that attaches to a motor 42. Motor 42 can be a single speed motor or a motor having varying speed capabilities.

Thus, motor 42 along with rotatable unit 40 constitutes a primary rotational means, which causes magnetic unit 22 and free moving member 30 to rotate about one axis. While the free moving member 30 rotates, not only does the magnetic unit 22 rotate according to this primary rotation means, but also according to the secondary rotation means created by the interaction of rolling member 26 and annular rolling surface 28.

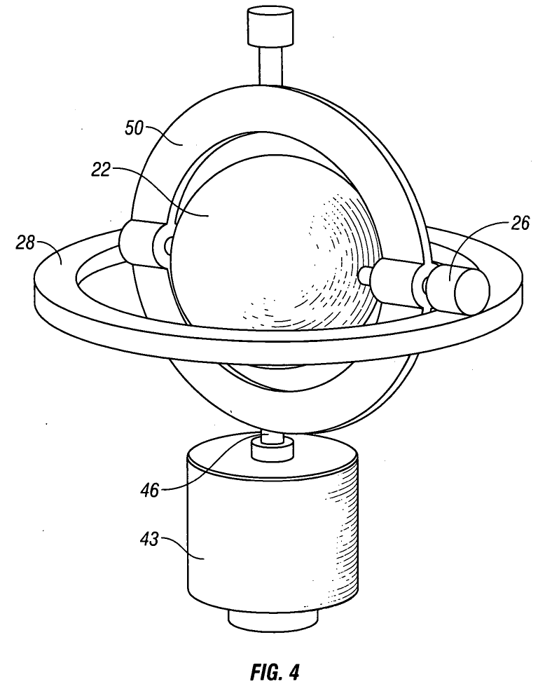

FIG. 4 represents yet another embodiment of the present invention. In this embodiment, a motor 43 is directly connected to the extension 46, with no need for a drive belt. The motor 43 turns the extension 46 directly, causing the free moving member 50 to rotate and having the same effect on the other parts in this embodiment as in the prior-disclosed embodiment.

FIG. 5 represents one type of cover 52 that may be used in conjunction with the presently preferred embodiments of the present invention. The cover 52 contains a power switch 56 connected to the motor (not shown). In the preferred embodiments, the cover is made of plastic, but can be made of other materials with similar generally non-magnetic properties. However, this invention can function independently without the use of the cover, or with the use of a partially transparent cover, such as window 54, so as to show the user the complex movement of the internal magnetic unit 22.

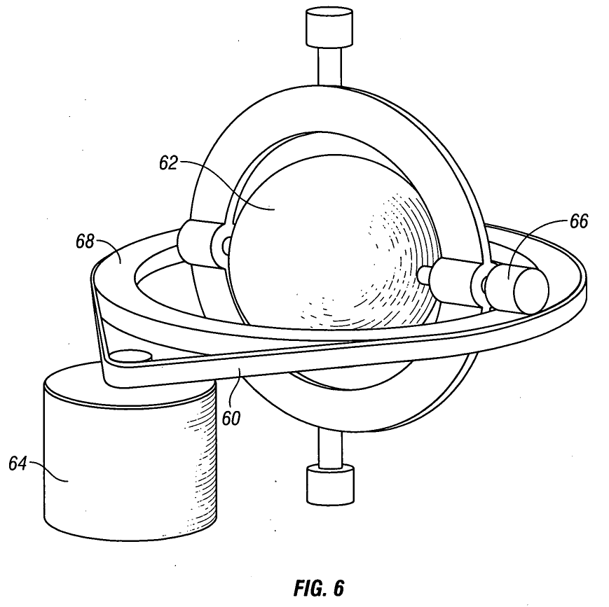

While FIGS. 1 and 2 illustrate one embodiment of the present invention in which gear teeth 9 and gear teeth 11 are on opposing surfaces of gear 8, other embodiments are equally contemplated by the present invention. For example, in FIG. 6, a motor 64 is in communication with the radially external surface of rotatable gear 68. As shown in FIG. 6, motor 64 causes drive belt 60 to turn gear 68, which is turn causes magnetic unit 62 to likewise turn. Magnetic unit 62 rotates as it turns because rotatable end means 66 is sandwiched between rotatable gear 68 and a fixed gear as described previously with respect to FIG. 2. The fixed gear is removed from view in FIG. 6 for purposes of clarity of the illustration. This fixed gear in combination with rotatable gear 68 causes rotatable end means 66 to rotate. As a result, magnetic unit 62, just as in FIGS. 1 and 2, rotates about two axes.

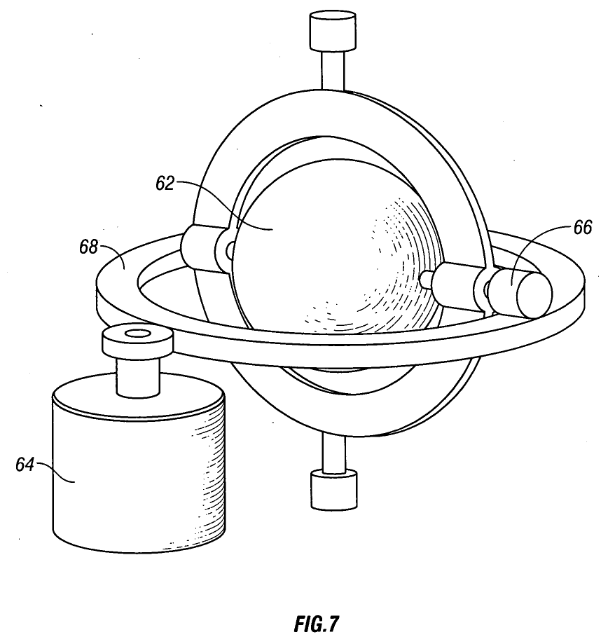

FIG. 6 illustrates an embodiment in which motor 64 is in communication with gear 68 indirectly, utilizing a drive belt 60. The drive belt shown is a rubberized drive belt, but could just as easily be a toothed belt, a chain, or the like, provided that the radially external surface of rotatable gear 68 comprises a matching gear, sprocket, or other friction features so that an angular force is exerted in the rotatable gear 68. Alternatively, motor 64 could be positioned so as to directly communicate with the radially external surface of gear 68 by way, of an orthogonal or beveled gear and tooth configuration, or other combination of gripping surfaces as shown in FIG. 7. The result in each instance is the same; magnetic unit 62 is caused to rotate in two axes at the same time thereby causing a complex bi-axial sweeping motion of the magnetic field emanating there from.

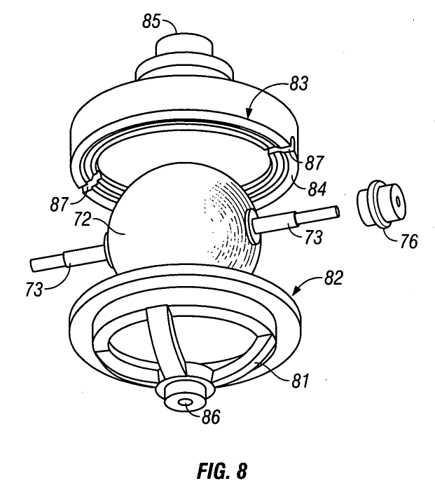

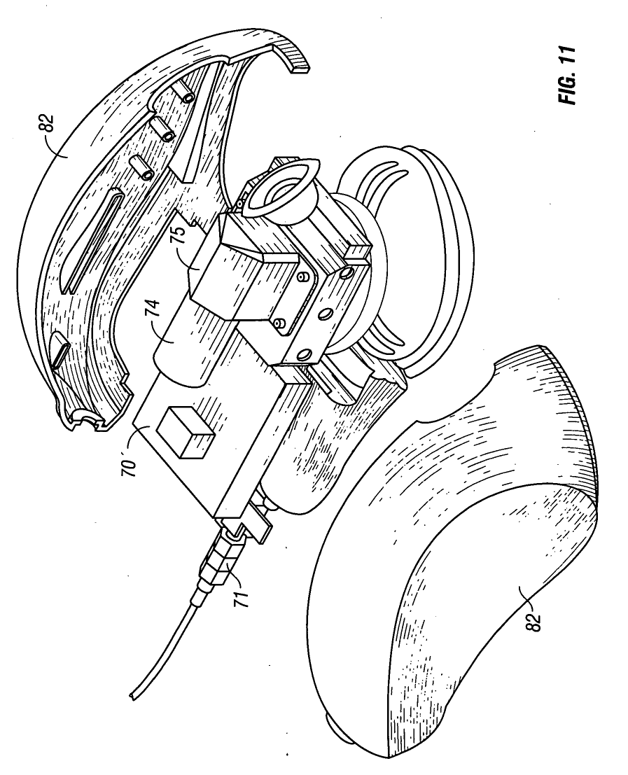

Another embodiment is shown in FIGS. 8 through 15. Like in the embodiments of FIG. 3, a motor 74 (shown in FIG. 11) exerts an angular force on magnetic unit 72 while annular rolling surface 78 remains fixed relative to cover 52. As a result, the magnetic unit 72 fixed to an end rolling member 76, as described in detail above with respect to rolling member 26, is forced to both rotate about a primary axis and at the same time roll about a secondary axis. In contrast to FIG. 3, the axis of rotation of the motor is perpendicular to the primary axis of rotation of the magnet assembly.

More particularly, the magnetic unit 72 is housed inside a free moving member 80 comprising a first half 81 and a second half 83. When mating surfaces 82 and 84 of the first and second halves of the free moving member 80, the inner surface of the free moving member 80 defines an approximately spherical chamber in which the magnetic unit 72 is held. With magnetic unit 72 placed in the chamber, mating surfaces 82 and 84 are then welded together at a sufficient number of places along to withstand the sort of impacts that are common to home appliances, such as being dropped during use, etc.

Mating surfaces 82 and 84 also define one or more clearances 87. When mating surfaces 82 and 84 are welded together, these clearances 87 define openings 88. The magnetic unit 72 further comprises two protruding arms 73 which extend away from magnetic unit 72 in diametrically opposite directions and extend through and beyond two openings 88. The arms could be separate pins or rods or the like extending from the magnetic unit 82, or they could alternatively be the distal ends of a single extending rod or pin. The present invention equally contemplates a magnetic unit 72 having only a single protruding arm or pin 73 provided the magnetic unit 72 remains sufficiently stable and free to rotate about an axis defined by the elongate length of said protruding arm 73. The chamber defined by the internal surfaces of the free moving member 80 and the openings 88 defined by the clearances 87 formed in mating surfaces 82 and 84 are both large enough to loosely retain magnetic unit 72 and protruding arms 73, respectively. Thus, while magnetic unit 72 is substantially enclosed within the free moving member 80, the magnetic unit 72 is capable of freely rotating relative to the free moving member 80 about the axis of rotation defined by the one or two protruding arms 73.

The free moving member 80 is held in an enclosure assembly 90. The enclosure assembly 90 comprises an inner surface 91 which defines an approximately spherical chamber, an annular rolling surface 78 formed in inner surface 91, and a pivot member 79 disposed in the inner surface 91. Also, a portion of the inner surface 91 functions as a wave washer retaining surface, as discussed further below. The enclosure assembly 90 is preferably fixed relative to the cover 52 and thus may be mounted to the cover or integrally formed in the inner surface of the cover 52. In either case, there is sufficient clearance between the outer surface of the free moving member 80 and the inner surface 91 of the enclosure assembly 90 so that the free moving member 80 may rotate about a pivot structure 86 which is formed in the first half 81 of free moving member 80 and which pivotally engages pivot member 79. FIG. 10 illustrates an embodiment that utilizes a bearing pin as pivot member 79. This function of pivotally mounting the free moving member 80 inside the enclosure assembly 90 may likewise be accomplished by other engaging structures, such as a circular recess formed in the enclosure coupled with a point formed at the apex of the first half 81 of the free moving member 80.

As mentioned above, in a preferred embodiment, the rotation of motor 74 is perpendicular to the desired rotation of the free moving member 80. The rotary motion of motor 74 is translated by a standard right-angle gear box 75, which comprises two mating angled gears or the like. At the same time, the speed of the motor may also be stepped up or down, which will inversely affect the torque of the imparted rotary motion. Presently, the best made involves a direct current or “DC” motor connected to a right-angle step-down gear box 75 for producing a ten-fold increase in torque. The DC motor is powered by a rechargeable battery 70 housed in battery case 70′ or directly from an AC/DC power converter 71 through plug-in jack 71′, which may also operate as a battery recharger as is common in home appliances.

The resultant rotary force of the motor, once translated 900 and stepped down by the gear box 75, drives free moving member 80 much the same way as illustrated in and discussed with respect to FIG. 4, above. The driving shaft (not shown) extends through axial opening 89 and directly imparts an angular force on drive extension 85 located at the apex of the second half 83 of free moving member 80.

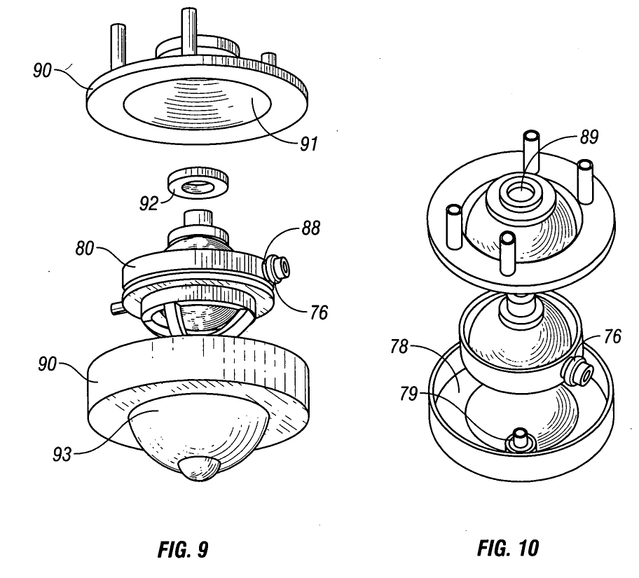

The end of one of the protruding arms 73 is equipped with rolling member 76. As free moving member 80 rotates, rolling member 76 is dragged along the annular rolling surface 78. Annular rolling surface 78 imparts an angular force on rolling member 76 as the latter moves along the annular rolling surface 78. A retainer member 92 is placed between the inner surface 91 of the enclosure assembly 90 and free moving member 80 to exert a downward axial force on free moving member 80. While the retainer member illustrated in FIG. 9 is a spring washer or wave-type washer, other means are equally contemplated for exerting an axial force on free moving member 80, such as a leaf spring, compression spring, diaphragm, or the like. This insures that a sufficient amount of contact occurs between rolling member 76 and annular rolling surface 78 to create this angular force on rolling member 76. As a result, rolling member 76 is forced to roll about the axis of its elongate dimension. A slipping member may be utilized in the opposite projecting arm 73, as described above in relation to FIGS. 3 and 4. With sufficiently tight tolerances between the magnet assembly and the enclosure assembly, however, a slipping member can be made to be unnecessary.

It is believed to be additionally advantageous to provide the operator with a visual means to realize the great degree of complex bi-axial movements that magnetic unit 72 is forced to make by this configuration. In FIG. 8, the first half 81 of free moving member 80 is shown as having large openings. This allows the complex movements of the magnetic unit 72 to be visible from outside the free moving member 80. It is equally contemplated that a transparent or semi-transparent material could be used to accomplish similar advantageous results. Similarly, the lower half 93 of the enclosure assembly 90 in FIG. 9 may be made from a transparent or semitransparent material, or be formed with relatively large openings as with first half 81. A window or lens 96, as shown in FIGS. 11 through 14, may form a part of cover 94.

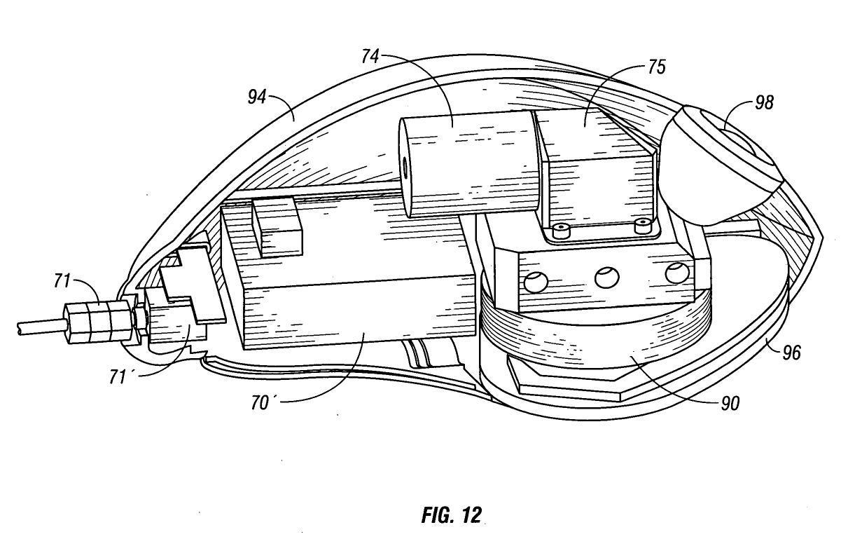

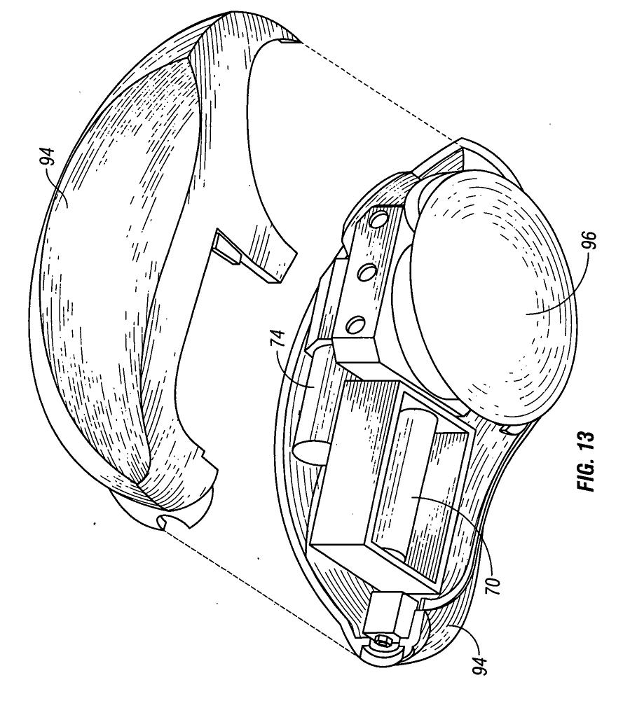

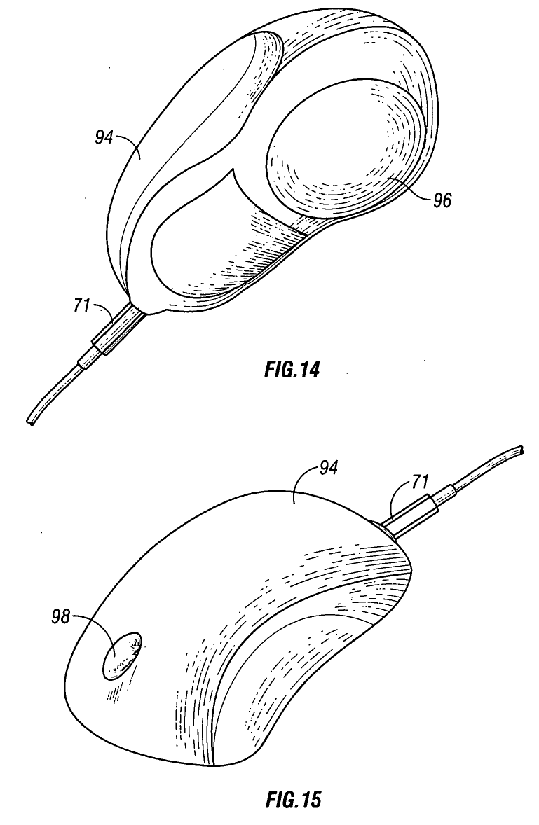

In addition to the window or lens 96, the configuration shown in FIGS. 12 and 13 provide a particularly efficient packaging of the present invention. The two halves of the cover 94 are plastic and molded to fit together and capture the window or lens 96, the power switch 98, the motor 74 and gear box 75, the battery case 70′, DC plug-in jack 71′, and the enclosure assembly 90. That is, essentially every component of the product is captured in one of the molded cover halves, the other half therefore being capable of removal without disrupting the arrangement of the components of the invention and product. FIGS. 12 and 13 show from the top and bottom, respectively, the efficient packing of the essential and auxiliary components for the present invention in the embodiment shown. FIGS. 14 and 15 show from the bottom and top, respectively, another cover contemplated by this invention. The power/recharge cord is detachable from the product and is not necessary for proper operation of the present invention, as the motor can run on batteries 70 as shown in FIG. 13.

Again, as mentioned above, all components other than the magnet unit are preferably made out of material that will not negatively affect the magnetic flux emanating from the magnetic unit 72. Instead, it is presently believed that the proper selection of materials for the enclosure assembly may be able to positively affect the magnetic flux by concentrating the magnetic energy and refocusing it towards the body part being treated.

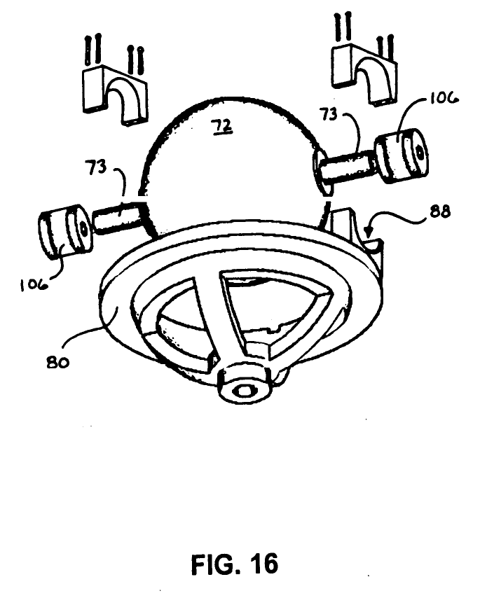

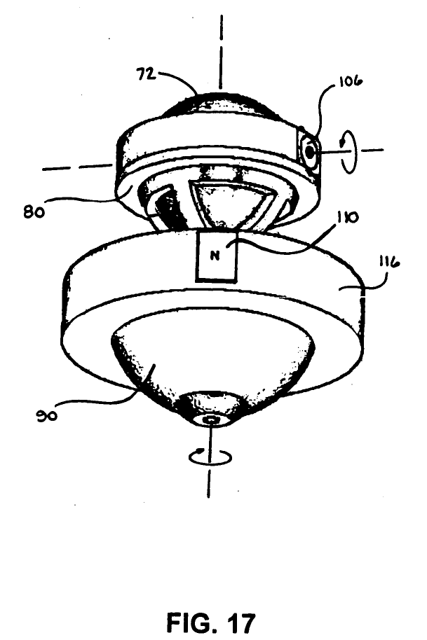

Another embodiment of the present invention is illustrated in FIGS. 16 and 17. In this embodiment, bi-axial rotation of the magnetic body is produced by introducing a stationary magnetic field oblique to the rotation of the magnetic body. The stationary magnetic field causes the rotating magnetic body to roll about an axis of second rotation that is oblique to its axis of first rotation. The embodiment illustrated in FIG. 17 comprises at least one stationary magnet 110 fixed relative to the stationary track 116, which may typically, but not necessarily, also be fixed relative to the enclosure assembly 90.

Magnetic body 72 rotates about an axis of first rotation in concert with free moving member 80, whereas the stationary magnet or magnets 110 do not rotate with free moving member 80. Magnetic body 72 is itself mounted rotatably within free moving member 80, such as by roller bearings 106 fixed to protruding arms 73 and mounted in openings 88 of enclosure assembly 90, and thereby can rotate about an axis of second rotation. When magnetic body 72 rotates about the axis of first rotation, it encounters the magnetic field emanating from the stationary magnets 110. This fixed magnetic field interacts with the rotating magnetic field of magnetic body 72, and thereby causes magnetic body 72 to rotate about the axis of second rotation without the magnetic body 72 or its protruding arms 73 ever making physical contact with stationary track 116.

In the embodiment shown in FIGS. 16 and 17, the stationary track 116 is slightly larger in diameter than the free moving member 80, and it preferably has 1 to 8 miniature surface button magnets attached to or embedded in the wall of the track preferably being evenly spaced around the track. Each stationary magnet 110 is preferably oriented such that one of its magnetic poles is pointed in the direction of the magnetic body 72. As a result, the effect of the magnetic flux of the stationary magnets 110 on magnetic body 72 is maximized. Alternatively, the stationary track 116 may be made entirely of a magnetic material that can be singularly magnetized in an orientation oblique to the axis of first rotation or magnetized in sections of alternating polarity to produce the same effect as the separately attached button magnets discussed above on the magnetic body 72.

Consequently, magnetic body 72 rotates either intermittently or constantly about the second axis of rotation due to its interactions with the magnetic flux of the stationary magnets 110 as the magnetic body 72 is forced to rotate about the first axis of rotation by the free moving member 80. This biaxial rotation occurs without magnetic body 72 ever making physical contact with the stationary track 116 but instead only engages magnetically with the track 116 by interacting with one or a combination of stationary magnetic fields. This embodiment therefore has the added advantage of reducing the point of physical contact and thus the number of parts that encounter friction and wear and tear. The embodiment is also somewhat less noisy due to this reduction in contacting parts.

Yet another embodiment of the present invention involves placing this magnet-to-magnet interaction effect at the surface of the outwardly extending roller member. That is, a magnetic roller member, in place of the roller member 76 shown in a number of the embodiments above, may extend beyond free moving member 80 and, as it rotates about the axis of first rotation, travel in a circle just above stationary track 116. In this embodiment, the magnetic roller member comprises one or more magnets that interact with one or more stationary magnets 110 along stationary track 116 to cause the magnetic roller members to roll about the axis of second rotation. This arrangement creates the necessary magnetic coupling to turn the magnetic body 72 about the axis of second rotation while it is being rotated about the axis of first rotation.

In this embodiment, each stationary magnet 110 is preferably oriented so that one of its magnetic poles is pointed in the direction of the magnetic roller member as it passes immediately overhead. As a result the magnetic effect of the stationary magnets 110 on the magnetic roller members is maximized, and the angular force on the magnetic body 72 to cause it to rotate about the axis of second rotation is thereby maximized.

FIG. 19 is an exploded perspective view of a magnetic therapeutic device 300 in accordance with an alternative embodiment of the present invention. Magnetic therapeutic device 300 includes a generally ring-shaped housing 302 adapted to rotatably accommodate a magnetic member 303. Magnetic member 303 is of a generally spherical configuration. Other suitable magnetic member configurations may be used, if needed. Magnetic member 303 may be made from permanent magnet material. Other suitable materials may be utilized to construct magnetic member 303, provided such other materials do not deviate from the intended scope of the present invention.

Magnetic member 303 is adapted to revolve on a central axle 305 within ring-shaped housing 302. Each end of central axle 305 is securely engaged by the wall of ring-shaped housing 302, as generally depicted in FIG. 19. The longitudinal axis of central axle 305 defines an axis of rotation 307 for magnetic member 303, as generally shown by rotational arrow 309 in FIG. 19.

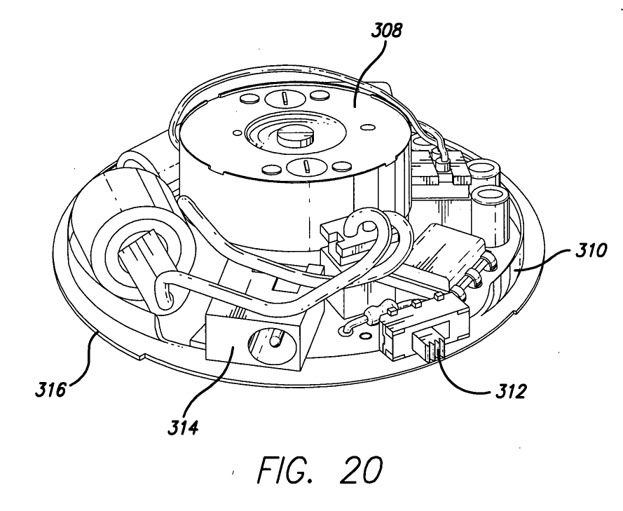

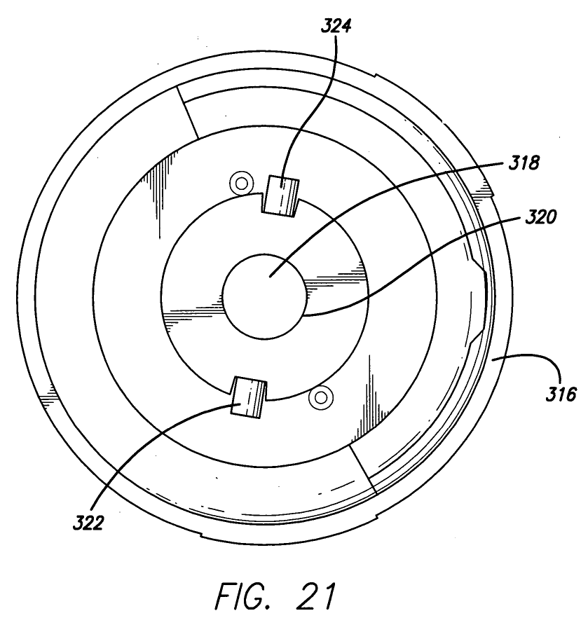

Ring-shaped housing 302 is equipped with a generally cylindrical bottom portion 304 adapted for coupling to a shaft 306 of motor 308. Motor 308 is operatively coupled to a printed circuit board (“PCB”) 310 which includes motor control circuitry and at least one power source. PCB 310 also includes an on/off actuator 312 and a power adapter 314. During assembly of magnetic therapeutic device 300, PCB 310 and motor 308 are housed within one side of a generally dome-shaped top cover member 316, as generally illustrated in FIG. 20. The opposite side of dome-shaped top cover member 316 includes an open interior 318 (FIG. 19) adapted to accommodate somewhat loosely ring-shaped housing 302 (with rotatably mounted magnetic member 303).

Once accommodated during assembly of magnetic therapeutic device 300, bottom portion 304 of ring-shaped housing 302 is coupled to motor shaft 306 through a generally circular bottom aperture 320 (FIG. 21) of dome-shaped top cover member 316. When operative, motor 308 causes ring-shaped housing 302 (with rotatably mounted magnetic member 303) to revolve about an axis of rotation 317 being defined by the longitudinal axis of motor shaft 306, as generally depicted by rotational arrow 315 in FIG. 19. A generally pan-like bottom cover member 321 (FIG. 19) is coupled to top cover member 316 to complete the assembly of magnetic therapeutic device 300.

Dome-shaped top cover member 316 is equipped with a pair of oppositely disposed permanent magnets 322, 324 (FIG. 21) on the side that is adapted to house motor 308 and PCB 310. Permanent magnet 322 is also generally shown in a partially cut-away view of dome-shaped top cover member 316 in FIG. 19. Other suitable materials and/or magnetic configurations may be used to provide the functionality of oppositely disposed permanent magnets 322, 324 (FIG. 21).

When magnetic therapeutic device 300 is assembled, as generally shown in FIG. 19, and motor actuator 312 is turned to an “on” position, oppositely disposed magnets 322, 324 cause magnetic member 303 to revolve about rotational axis 307 while magnetic member 303 with ring-shaped housing 302 collectively revolve about rotational axis 317. When magnetic therapeutic device 300 is operational, the biaxial rotation of magnetic member 303 in conjunction with the single-axle rotation of ring-shaped housing 302 generates vibration that may provide therapeutic massage-like effects to a treatment area of a human/animal body. This type of therapy may be readily combined with conventional heat and/or vibrational therapy, as needed.

A person skilled in the art would appreciate that exemplary embodiments described hereinabove are merely illustrative of the general principles of the present invention. Other modifications or variations may be employed that are within the scope of the invention. Thus, by way of example, but not of limitation, alternative configurations may be utilized in accordance with the teachings herein. Accordingly, the drawings and description are illustrative and not meant to be a limitation thereof.

Moreover, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Thus, it is intended that the invention cover all embodiments and variations thereof as long as such embodiments and variations come within the scope of the appended claims and their equivalents.

1. A magnetic therapeutic device, comprising:
a housing adapted to revolve about a first axis of rotation; and
at least one magnetic member adapted to revolve about a second axis of rotation within said housing while said housing revolves about said first axis of rotation, said at least one magnetic member simultaneously revolving about said first and second axes to provide therapeutic massage-like effects to a treatment area.

2. The magnetic therapeutic device of claim 1, further comprising a top cover member having a first side being equipped with a pair of oppositely disposed permanent magnets, said oppositely disposed permanent magnets being operatively coupled to said at least one magnetic member.

3. The magnetic therapeutic device of claim 2, wherein said first side is adapted to house at least one printed circuit board (“PCB”) and at least one motor with a shaft, said at least one motor adapted to revolve said housing about said first axis.

4. The magnetic therapeutic device of claim 3, wherein said pair of oppositely disposed permanent magnets cause said at least one magnetic member to revolve about said second axis within said housing while said housing revolves about said first axis of rotation.

5. The magnetic therapeutic device of claim 3, wherein said PCB includes motor circuitry and at least one motor power source.

6. The magnetic therapeutic device of claim 5, wherein said PCB further includes an on/off actuator and a power adapter.

7. The magnetic therapeutic device of claim 3, wherein said motor is operatively coupled to said PCB.

8. The magnetic therapeutic device of claim 1, wherein said at least one magnetic member has substantially spherical configuration.

9. The magnetic therapeutic device of claim 8, wherein said at least one substantially spherical magnetic member is made from permanent magnet material.

10. The magnetic therapeutic device of claim 9, wherein said housing has substantially ring-shaped configuration.

11. The magnetic therapeutic device of claim 10, wherein said at least one substantially spherical magnetic member is adapted to revolve on a central axle within said substantially ring-shaped housing, said central axle having a longitudinal axis.

12. The magnetic therapeutic device of claim 11, wherein each end of said central axle is securely engaged by the wall of said substantially ring-shaped housing.

13. The magnetic therapeutic device of claim 11, wherein said longitudinal axis of said central axle defines said second axis of rotation.

14. The magnetic therapeutic device of claim 3, wherein said motor shaft has a longitudinal axis defining said first axis of rotation.

15. The magnetic therapeutic device of claim 2, wherein said top cover member has a second side adapted to rotatably accommodate said housing.

16. The magnetic therapeutic device of claim 2, wherein said top cover member is substantially dome-shaped.

17. The magnetic therapeutic device of claim 16, further comprising a bottom cover member adapted for coupling to said top cover member.

18. The magnetic therapeutic device of claim 17, wherein said bottom cover member has substantially pan-like configuration.
